(19) United States
(12) Patent Application Publication
(10) Pub. No.: US 2022/0080023 A1
(43) Pub. Date: Mar. 17, 2022

(54) NOVEL COMPOSITION

(71) Applicant: ARECOR LIMITED, Saffron Walden (GB)

(72) Inventors: Jan JEZEK, Saffron Walden (GB); David GERRING, Saffron Walden (GB); Sarah HOWELL, Saffron Walden (GB); Jorge PINTO, Saffron Walden (GB)

(21) Appl. No.: 17/416,845

(22) PCT Filed: Dec. 20, 2019
(86) PCT No.: PCT/GB2019/053645

§ 371 Date: Jun. 21, 2021

(30) Foreign Application Priority Data
Dec. 21, 2018 (GB) 1821148.2
Jun. 27, 2019 (GB) 1909264.2

(51) Int. Cl. A61K 38/095; A61K 47/12; A61K 47/18; A61K 47/02; A61K 47/20; A61K 47/22; A61K 47/26

(52) U.S. Cl. CPC A61K 38/095; A61K 47/12; A61K 47/183; A61K 47/26; A61K 47/20; A61K 47/22; A61K 47/02

(57) ABSTRACT

The invention provides inter alia an aqueous solution composition of pH in the range 4.0-6.0 comprising: —terlipressin or a salt thereof; —optionally one or more buffers being substances having at least one ionisable group with a pKa in the range 3.0 to 7.0 and which pKa is within 1 pH unit of the pH of the composition; —optionally an amino acid; and—optionally a tonicity modifier wherein the buffers are present in the composition at a total concentration of 0-5 mM.

This invention relates to aqueous solution compositions of terlipressin, at low buffer concentrations.

### BACKGROUND

Terlipressin is a synthetic analogue of vasopressin, a peptide hormone which has important antidiuretic and vasopressor actions and a variety of other actions including glycogenolysis. Terlipressin is used as a vasoactive drug in the management of low blood pressure, and is also used to treat norepinephrine-resistant septic shock, bleeding oesophageal varices, and in the treatment of hepatorenal syndrome. Terlipressin is generally administered via intravenous (IV) injection.

Terlipressin is a cyclic peptide, the structure of which is shown in FIG. 1. When formulated as aqueous solutions, such peptides therapeutics tend to be unstable and are susceptible to structural degradation and consequent loss of biological activity while stored. The structural degradation is typically chemical in nature, including hydrolytic cleavage, cyclic imide formation, isomerization or oxidation. In some cases, the degradation can be physical in nature (e.g. aggregation or gel formation), although these processes are relatively less common in peptides than in the case of larger proteins.

The rates of the degradation processes are proportional to temperature, and peptide therapeutics such as terlipressin are generally more stable at lower temperatures. However, to ensure convenience for patients there is often need to develop products that are stable at elevated temperatures, such as up to 25° C. or up to 30° C., either for a specific period of time or for the entire shelf-life.

One of the most critical parameters to control the stability of peptide therapeutics such as terlipressin is pH. Therefore, pH optimization is a key step in formulation development. Many therapeutic peptides are formulated at a selected pH range, for example, at pH between 4.0-6.0. It is thought to be important to ensure that the pH is maintained at the selected range and pH fluctuations are minimized. Therefore, it has been understood that a certain degree of buffering capacity is needed in the formulation. Whilst some peptide therapeutic agents provide buffering capacity themselves in the selected pH range (i.e. are self-buffering), others such as terlipressin do not.

The present invention addresses the problem of instability of terlipressin when formulated in an aqueous solution composition.

WO2006/138181A2 (AMGEN, INC.) discloses self-buffering protein formulations which are substantially free of other buffering agents.

US2016/0106840A1 (EAGLE PHARMACEUTICALS INC.) discloses bivalirudin compositions which have a pH of about 3 to about 5 and are substantially free of buffer.

U.S. Pat. No. 8,420,081B2 (ABBVIE INC.) discloses aqueous formulations of antibodies wherein the formulations have conductivity of less than about 2.5 mS/cm.

WO2008/084237 (ARECOR LIMITED) discloses protein compositions which do not comprise conventional buffers in a meaningful amount. Instead “displaced buffers” which are additives with pKₐ values at least 1 unit less than or 1 unit greater than the pH of the composition are utilised.

### SUMMARY OF THE INVENTION

Thus, according to the present invention, there is provided an aqueous solution composition of pH in the range 4.0 to 6.0 comprising:
terlipressin or a salt thereof;
optionally one or more buffers being substances having at least one ionisable group with a pKₐ in the range 3.0 to 7.0 and which pKₐ is within 1 pH unit of the pH of the composition;
optionally an amino acid; and
optionally a tonicity modifier;
   wherein the buffers are present in the composition at a total concentration of 0-5 mM.

### FIGURES

FIG. 1: Structure of terlipressin.

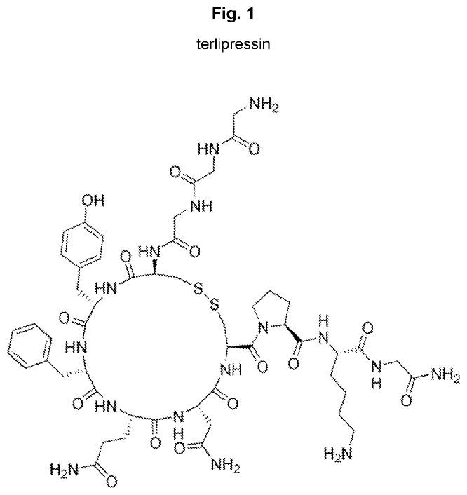

### DETAILED DESCRIPTION OF THE INVENTION

Described herein are stable aqueous solution compositions of terlipressin having no or very low concentration of buffer.

It should be noted that all references herein to “pH” refer to the pH of a composition evaluated at 25° C. All references to “pKₐ” refer to the pKₐ of an ionisable group evaluated at 25° C. (see CRC Handbook of Chemistry and Physics, 79^{th} Edition, 1998, D. R. Lide).

The pH of the composition of the invention in the range 4.0 to 6.0, such as 4.5 to 5.5, such as 4.5 to 5. In one embodiment, the pH of the composition is about 4.5. In one embodiment, the pH of the composition is about 5.

The present inventors have discovered that buffers have a detrimental impact on the stability of terlipressin, in a concentration-dependent manner. Therefore, the concentration of buffer in the composition should be limited as much as possible.

In an embodiment, the composition does not contain a buffer. In an embodiment, the composition contains a single buffer. In an embodiment, the composition contains two buffers.

In one embodiment, the total concentration of buffers in the solution is 0-5 mM such as 0-4 mM, 0-3 mM, 0-2 mM, 0-1 mM, 0-0.5 mM, 0-0.4 mM, 0-0.3 mM, 0-0.2 mM or 0-0.1 mM. In one embodiment, the total concentration of buffers in the solution is 0-4 mM, such as 0-3 mM, 0-2 mM, 0-1 mM, 0-0.5 mM, 0-0.4 mM, 0-0.3 mM, 0-0.2 mM or 0-0.1 mM. In one embodiment, the total concentration of buffers in the solution is 0-3 mM, such as 0-2 mM, 0-1 mM, 0-0.5 mM, 0-0.4 mM, 0-0.3 mM, 0-0.2 mM or 0-0.1 mM. In one embodiment, the total concentration of buffers in the solution is 0-2 mM, such as 0-1 mM, 0-0.5 mM, 0-0.4 mM, 0-0.3 mM, 0-0.2 mM or 0-0.1 mM. In one embodiment, the total concentration of buffers in the solution is 0-1 mM, such as 0-0.5 mM, 0-0.4 mM, 0-0.3 mM, 0-0.2 mM or 0-0.1 mM.

In one embodiment, the total concentration of buffers in the solution is 0.1-5 mM, such as 0.1-4 mM, 0.1-3 mM, 0.1-2 mM, 0.1-1 mM, 0.1-0.5 mM, 0.1-0.4 mM, 0.1-0.3 mM or 0.1-0.2 mM. In one embodiment, the total concentration of buffers in the solution is 0.1-4 mM, such as 0.1-3 mM, 0.1-2 mM, 0.1-1 mM, 0.1-0.5 mM, 0.1-0.4 mM, 0.1-0.3 mM or 0.1-0.2 mM. In one embodiment, the total concentration of buffers in the solution is 0.1-3 mM, such as 0.1-2 mM, 0.1-1 mM, 0.1-0.5 mM, 0.1-0.4 mM, 0.1-0.3 mM or 0.1-0.2 mM. In one embodiment, the total concentration of buffers in the solution is 0.1-2 mM, such as 0.1-1 mM, 0.1-0.5 mM, 0.1-0.4 mM, 0.1-0.3 mM or 0.1-0.2 mM. In one embodiment, the total concentration of buffers in the solution is 0.1-1 mM, such as 0.1-0.5 mM, 0.1-0.4 mM, 0.1-0.3 mM or 0.1-0.2 mM.

In one embodiment, the total concentration of buffers in the solution is 0.2-5 mM such as 0.2-4 mM, 0.2-3 mM, 0.2-2 mM, 0.2-1 mM, 0.2-0.5 mM, 0.2-0.4 mM or 0.2-0.3 mM. In one embodiment, the total concentration of buffers in the solution is 0.2-4 mM, such as 0.2-3 mM, 0.2-2 mM, 0.2-1 mM, 0.2-0.5 mM, 0.2-0.4 mM or 0.2-0.3 mM. In one embodiment, the total concentration of buffers in the solution is 0.2-3 mM, such as 0.2-2 mM, 0.2-1 mM, 0.2-0.5 mM, 0.2-0.4 mM or 0.2-0.3 mM. In one embodiment, the total concentration of buffers in the solution is 0.2-2 mM, such as 0.2-1 mM, 0.2-0.5 mM, 0.2-0.4 mM or 0.2-0.3 mM. In one embodiment, the total concentration of buffers in the solution is 0.2-1 mM, such as 0.2-0.5 mM, 0.2-0.4 mM or 0.2-0.3 mM.

In one embodiment, the total concentration of buffers in the solution is 0.3-5 mM, such as 0.3-4 mM, 0.3-3 mM, 0.3-2 mM, 0.3-1 mM, 0.3-0.5 mM or 0.3-0.4 mM. In one embodiment, the total concentration of buffers in the solution is 0.3-4 mM, such as 0.3-3 mM, 0.3-2 mM, 0.3-1 mM, 0.3-0.5 mM or 0.3-0.4 mM. In one embodiment, the total concentration of buffers in the solution is 0.3-3 mM, such as 0.3-2 mM, 0.3-1 mM, 0.3-0.5 mM or 0.3-0.4 mM. In one embodiment, the total concentration of buffers in the solution is 0.3-2 mM, such as 0.3-1 mM, 0.3-0.5 mM or 0.3-0.4 mM. In one embodiment, the total concentration of buffers in the solution is 0.3-1 mM, such as 0.3-0.5 mM or 0.3-0.4 mM.

In one embodiment, the total concentration of buffers in the solution is 0.4-5 mM, such as 0.4-4 mM, 0.4-3 mM, 0.4-2 mM, 0.4-1 mM or 0.4-0.5 mM. In one embodiment, the total concentration of buffers in the solution is 0.4-4 mM such as 0.4-3 mM, 0.4-2 mM, 0.4-1 mM or 0.4-0.5 mM. In one embodiment, the total concentration of buffers in the solution is 0.4-3 mM, such as 0.4-2 mM, 0.4-1 mM or 0.4-0.5 mM. In one embodiment, the total concentration of buffers in the solution is 0.4-2 mM, such as 0.4-1 mM or 0.4-0.5 mM. In one embodiment, the total concentration of buffers in the solution is 0.4-1 mM or 0.4-0.5 mM.

In one embodiment, the total concentration of buffers in the solution is 0.5-5 mM, such as 0.5-4.5 mM, 0.5-4 mM, 0.5-3 mM, 0.5-2 mM or 0.5-1 mM. In one embodiment, the total concentration of buffers in the solution is 0.5-4 mM, such as 0.5-3 mM, 0.5-2 mM or 0.5-1 mM. In one embodiment, the total concentration of buffers in the solution is 0.5-3 mM, such as 0.5-2 mM or 0.5-1 mM. In one embodiment, the total concentration of buffers in the solution is 0.5-2 mM or 0.5-1 mM.

In one embodiment, the total concentration of buffers in the composition is ≤5 mM, such as ≤4 mM, ≤3 mM, ≤2 mM, ≤1 mM, ≤0.5 mM, ≤0.4 mM, ≤0.3 mM, ≤0.2 mM ≤0.1 mM.

In one embodiment, the composition is substantially free of buffer. For the avoidance of doubt, when considering the concentration of buffer in the composition, any buffering capacity of the terlipressin itself should be excluded.

The pH of an aqueous solution decreases if an acid is added and increases if a base is added. At a given temperature and atmospheric pressure, the magnitude of the pH decrease on addition of an acid or the magnitude of the pH increase on addition of a base depends on (1) the amount of the acid or the base added, (2) the starting pH of the aqueous solution (i.e. prior to the addition of the acid or the base) and (3) the presence of a buffer. Thus, (1) starting from a given pH, the addition of a greater amount of an acid or a base will result in greater magnitude of pH change, (2) addition of a given amount of an acid or a base will result in the greatest pH change at neutral pH (i.e. pH 7.0) and the magnitude of the pH change will decrease as the starting pH moves away from pH 7.0 and (3) the magnitude of the pH change, starting from a given pH, will be smaller in the presence of a buffer than in the absence of a buffer. A buffer thus has the ability to reduce the change in pH if an acid or a base is added to the solution.

Suitably, a substance is considered to be a buffer if it is capable of reducing the magnitude of the pH change of a solution to 75%, preferably 50%, most preferably to 25%, compared with an identical solution that does not comprise the buffer, when either strong acid or a strong base is added resulting in 0.1 mM increase of the acid or the base in the solution.

Conversely, suitably, a substance is not considered to be a buffer if it is not capable of reducing the magnitude of the pH change of a solution to 75%, preferably 50%, most preferably to 25%, compared with an identical solution that does not comprise the substance, when either strong acid or a strong base is added resulting in 0.1 mM increase of the acid or the base in the solution.

In one embodiment, the or a buffer is an amino acid. In another embodiment, the or a buffer is not an amino acid.

In one embodiment, the composition comprises a buffer or buffers selected from the group consisting of histidine, maleate, glyoxylate, aspartame, glucuronate, aspartate, glutamate, tartrate, gluconate, lactate, glycolic acid, adenine, succinate, ascorbate, benzoate, phenylacetate, gallate, cytosine, p-aminobenzoic acid, sorbate, acetate, propionate, alginate, urate, 2-(N-morpholino)ethanesulphonic acid, bicarbonate, bis(2-hydroxyethyl) iminotris(hydroxymethyl)methane, N-(2-acetamido)-2-iminodiacetic acid, 2-[(2-amino-2-oxoethyl)amino]ethanesulphonic acid, piperazine and N,N′-bis(2-ethanesulphonic acid). Such buffers contain ionisable groups with a pKₐ in the range 3.0 to 7.0. In one embodiment, the buffer is selected from the group consisting of histidine, maleate, tartrate, lactate, benzoate, acetate and bicarbonate. In one embodiment the buffer is an organic mono-carboxylic acid e.g. an aliphatic mono-carboxylic acid such as acetate or lactate or an organic di-carboxylic acid e.g. an aliphatic di-carboxylic acid such as succinate, tartrate, malate or maleate or an aromatic mono-carboxylic acid such as benzoate. In one embodiment, the buffer comprises succinate buffer or is succinate buffer. In one embodiment the buffer comprises benzoate buffer or is benzoate buffer. In one embodiment the buffer comprises lactate buffer or is lactate buffer. In one embodiment the buffer comprises acetate buffer or is acetate buffer. Suitably the buffer is not citrate i.e. the composition of the invention does not comprise citrate. As shown in Example 2, citrate was found to have a destabilising effect on compositions. As shown in Example 2, compositions containing a low concentration of acetate or lactate as buffer appeared to have particularly good stability, particularly acetate as buffer.

In one embodiment, the composition of the invention comprises ≤1 mM, such as ≤2 mM, ≤3 mM or ≤4 mM acetate ions. In this regard, it should be noted that although terlipressin may be employed in the form of an acetate salt, the concentration of acetate ions would suitably be sufficiently low so as to have insignificant buffering capacity (such as <2 mM or <1 mM). In one embodiment, the composition of the invention does not contain acetate ions. In one embodiment the composition of the invention does not contain acetate ions save those which are present as a counter ion to terlipressin.

It should be noted that when calculating the concentration of buffer in the aqueous solution composition, any counterions present in salt forms of terlipressin with buffering capacity should be included in the total concentration of buffer.

The concentration of terlipressin in the composition is typically 0.001-50 mg/ml, such as 0.01-10 mg/ml, 0.01-5 mg/ml, 0.01-3 mg/ml, 0.01-2 mg/ml, 0.01-1 mg/ml or 0.1-1 mg/ml.

Salts of terlipressin may be formed with a suitable counter ion, suitably a pharmaceutically acceptable counter ion, including, but not limited to, acetate, benzoate, chloride, lactate, nitrate, sulfate, maleate and succinate. Suitably terlipressin is employed as terlipressin acetate.

When the counterion of terlipressin is a buffer (e.g. the counterion is acetate), in an embodiment: (i) the composition contains no further buffer (e.g. no further acetate); or (ii) composition contains a further amount of the same buffer (e.g. further acetate); or (iii) composition also contains an amount of a different buffer (e.g. a buffer other than acetate).

The present inventors have discovered that as well as minimising buffer concentration, in certain embodiments the addition of certain additives to the composition can provide further stability benefits.

At certain pH ranges the additives described herein may have buffering capacity, e.g. in a composition of pH in the range 4.0-6.0, the stabilizing additives may have at least one ionisable group with a pKₐ in the range 3.0 to 7.0 and which pKₐ is within 1 pH unit of the pH of the composition. For the avoidance of doubt, the concentration of such stabilizing additives should be included in the total concentration of buffers. Such stabilizing additives are therefore present in the composition at a concentration which is sufficiently low such that the total buffer concentration limitation is met e.g. does not exceed 5 mM.

In one embodiment, the composition further comprises an amino acid, particularly a natural amino acid, such as an α-amino acid. As shown in Example 3, the inclusion of an amount of amino acid in the composition is capable of increasing the stability of the composition. In one embodiment, an amino acid is selected from the group consisting of methionine, glycine, proline, arginine, lysine, aspartic acid, glutamic acid and histidine e.g. is selected from the group consisting of methionine, glycine, proline, arginine, lysine and aspartic acid; and in particular is selected from the group consisting of methionine, glycine, proline, arginine and lysine. In one embodiment, the amino acid is methionine. In one embodiment, the amino acid is glycine. In one embodiment, the amino acid is proline.

Some amino acids (such as glycine or arginine) may also serve the purpose of being a charged tonicity modifier as discussed below.

In one embodiment, the amino acid is not a buffer at the given pH of the composition i.e. the amino acid does not comprise ionisable groups with pKₐ within 1 pH unit of the pH of the composition.

The amino acid is present in a concentration sufficient to provide a stabilizing effect. In one embodiment, the amino acid is present at a concentration of 1-200 mM, such as 1-100 mM, 1-50 mM, 1-20 mM, 1-10 mM, 1-5 mM, 1-4 mM, 1-3 mM or 1-2 mM. As shown in Example 3, suitably the concentration of the amino acid is not too high. In one embodiment, the amino acid is present at a concentration of 2-20 mM, 2-10 mM or 5-10 mM e.g. around 5 mM.

In one embodiment, an amino acid is present in the composition and is selected from aspartic acid, glutamic acid and histidine, at a concentration of 5 mM or less.

The composition may comprise a tonicity modifier, which may be charged or uncharged. In certain embodiments, the tonicity modifier, particularly an uncharged tonicity modifier, can have a stabilizing effect. Examples of uncharged tonicity modifiers include sugars (such as sucrose, trehalose and lactose), sugar alcohols (such as mannitol and sorbitol), other polyols (such as glycerol and 1,2-propanediol) and polyethylene glycols (such as PEG300 and PEG400). In one embodiment, the uncharged tonicity modifier is selected from the group consisting of glycerol, 1,2-propanediol, mannitol, sorbitol, sucrose, trehalose, lactose, PEG300 and PEG400. In one embodiment, the uncharged tonicity modifier is selected from the group consisting of mannitol and sorbitol. When included, an uncharged tonicity modifier is typically employed in the composition at a concentration of 50-1000 mM, for example 200-500 mM, such as about 300 mM. Examples of charged tonicity modifiers include sodium chloride, sodium sulphate, and amino acids such as glycine or arginine. In one embodiment, the charged tonicity modifier is selected from the group consisting of sodium chloride, sodium sulphate, and amino acids such as glycine or arginine, and in particular selected from sodium chloride and sodium sulphate. In one embodiment, the charged tonicity modifier is sodium chloride. Amino acids that have buffering capacity within the pH range of the composition are suitably employed at a concentration which is sufficiently low such that the total buffer concentration does not exceed 5 mM. When included, a charged tonicity modifier is typically employed in the composition at a concentration of 25-500 mM, for example 50-250 mM such as about 150 mM; or at a concentration of 0.1-5 mM such as 0.1-4 mM, 0.1-3 mM, 0.1-2 mM or 0.1-1 mM.

The compositions of the invention may have a wide range of osmolarity values, including hypotonic, isotonic and hypertonic compositions. Suitably the compositions of the invention are substantially isotonic. In one embodiment, the compositions of the invention are isotonic. Suitably, the osmolarity of the compositions of the invention is selected to minimize pain according to the route of administration e.g. upon injection. Suitable compositions of the invention when intended for direct administration have an osmolarity in the range of about 200 mOsm/L to about 500 mOsm/L. Suitably, the osmolarity is in the range of about 250 mOsm/L to about 350 mOsm/L. More suitably, the osmolarity is about 300 mOsm/L.

The composition may comprise a non-ionic surfactant.

A particularly suitable class of non-ionic surfactants is the polysorbates (fatty acid esters of ethoxylated sorbitan), such as polysorbate 20 or polysorbate 80. Polysorbate 20 is a mono ester formed from lauric acid and polyoxyethylene (20) sorbitan in which the number 20 indicates the number of oxyethylene groups in the molecule. Polysorbate 80 is a mono ester formed from oleic acid and polyoxyethylene (20) sorbitan in which the number 20 indicates the number of oxyethylene groups in the molecule. Polysorbate 20 is known under a range of brand names including in particular Tween 20, and also Alkest TW 20. Polysorbate 80 is known under a range of brand names including in particular Tween 80, and also Alkest TW 80. Other suitable polysorbates include polysorbate 40 and polysorbate 60.

Another suitable class of non-ionic surfactants is the alkyl glycosides, especially dodecyl maltoside. Other alkyl glycosides include dodecyl glucoside, octyl glucoside, octyl maltoside, decyl glucoside, decyl maltoside, tridecyl glucoside, tridecyl maltoside, tetradecyl glucoside, tetradecyl maltoside, hexadecyl glucoside, hexadecyl maltoside, sucrose monooctanoate, sucrose mono decanoate, sucrose monododecanoate, sucrose monotridecanoate, sucrose monotetradecanoate and sucrose monohexadecanoate.

Another suitable class of non-ionic surfactants is block copolymers of polyethylene glycol and polypropylene glycol, also known as poloxamers, especially poloxamer 188, poloxamer 407, poloxamer 171 and poloxamer 185. Poloxamers are also known under brand names Pluronics or Koliphors. For example, poloxamer 188 is marketed as Pluronic F-68.

Another suitable class of non-ionic surfactants is alkyl ethers of polyethylene glycol, especially those known under a brand name Brij, such as selected from polyethylene glycol (2) hexadecyl ether (Brij 52), polyethylene glycol (2) oleyl ether (Brij 93) and polyethylene glycol (2) dodecyl ether (Brij L4). Other suitable Brij surfactants include polyethylene glycol (4) lauryl ether (Brij 30), polyethylene glycol (10) lauryl ether (Brij 35), polyethylene glycol (20) hexadecyl ether (Brij 58) and polyethylene glycol (10) stearyl ether (Brij 78).

Another suitable class of non-ionic surfactants are alkylphenyl ethers of polyethylene glycol, especially 4-(1,1,3,3-tetramethylbutyl)phenyl-polyethylene glycol, also known under a brand name Triton X-100.

In one embodiment, the non-ionic surfactant is selected from the group consisting of an alkyl glycoside, a polysorbate, an alkyl ether of polyethylene glycol, a block copolymer of polyethylene glycol and polypropylene glycol, and an alkylphenyl ether of polyethylene glycol.

In one embodiment, the non-ionic surfactant is a polysorbate or a poloxamer, and is suitably a polysorbate such as polysorbate 20 or polysorbate 80. The concentration of the non-ionic surfactant in the composition will typically be in the range 10-2000 μg/ml, such as 50-1000 μg/ml, 100-500 μg/ml or about 200 μg/ml.

The compositions of the invention may additionally comprise a preservative such as a phenolic or a benzylic preservative. The preservative is suitably selected from the group consisting of phenol, m-cresol, chlorocresol, benzyl alcohol, propyl paraben and methyl paraben, in particular phenol, m-cresol and benzyl alcohol. The concentration of preservative is typically 10-100 mM, for example 20-80 mM, such as 25-50 mM. The optimal concentration of the preservative in the composition is selected to ensure the composition passes the Pharmacopoeia Antimicrobial Effectiveness Test (USP <51>, Vol. 32).

In one embodiment, there is provided an aqueous solution composition of pH in the range 4.0 to 6.0 comprising or consisting of:
terlipressin or a salt thereof;
optionally one or more buffers being substances having at least one ionisable group with a pKₐ in the range 3.0 to 7.0 and which pKₐ is within 1 pH unit of the pH of the composition;
an amino acid;
a tonicity modifier;
optionally one or more preservatives; and
optionally one or more non-ionic surfactants;
wherein the buffers are present in the composition at a total concentration of 0-5 mM, such as 0-4 mM, 0-3 mM, 0-2 mM, 0-1 mM, 0-0.5 mM, 0-0.4 mM, 0-0.3 mM, 0-0.2 mM or 0-0.1 mM; or at a total concentration of 0.1-5 mM, such as 0.1-4 mM, 0.1-3 mM, 0.1-2 mM, 0.1-1 mM, 0.1-0.5 mM, 0.1-0.4 mM, 0.1-0.3 mM or 0.1-0.2 mM; or at a total concentration of 0.2-5 mM, such as 0.2-4 mM, 0.2-3 mM, 0.2-2 mM, 0.2-1 mM, 0.2-0.5 mM, 0.2-0.4 mM or 0.2-0.3 mM; or at a total concentration of 0.3-5 mM, such as 0.3-4 mM, 0.3-3 mM, 0.3-2 mM, 0.3-1 mM, 0.3-0.5 mM or 0.3-0.4 mM; or at a total concentration of 0.4-5 mM, such as 0.4-4 mM, 0.4-3 mM, 0.4-2 mM, 0.4-1 mM or 0.4-0.5 mM; or at a total concentration of 0.5-5 mM, such as 0.5-4 mM, 0.5-3 mM, 0.5-2 mM or 0.5-1 mM.

In one embodiment, there is provided an aqueous solution composition of pH in the range 4.0 to 6.0 comprising or consisting of:
terlipressin or a salt thereof;
optionally one or more buffers being substances having at least one ionisable group with a pKₐ in the range 3.0 to 7.0 and which pKₐ is within 1 pH unit of the pH of the composition;
an amino acid selected from the group consisting of methionine, glycine, proline, arginine, lysine, aspartic acid, glutamic acid and histidine, in particular selected from the group consisting of methionine, glycine, proline, arginine and lysine, e.g. glycine, proline or methionine e.g. methionine;
a tonicity modifier;
optionally one or more preservatives; and
optionally one or more non-ionic surfactants;
   wherein the buffers are present in the composition at a total concentration of 0-5 mM, such as 0-4 mM, 0-3 mM, 0-2 mM, 0-1 mM, 0-0.5 mM, 0-0.4 mM, 0-0.3 mM, 0-0.2 mM or 0-0.1 mM; or at a total concentration of 0.1-5 mM, such as 0.1-4 mM, 0.1-3 mM, 0.1-2 mM, 0.1-1 mM, 0.1-0.5 mM, 0.1-0.4 mM, 0.1-0.3 mM or 0.1-0.2 mM; or at a total concentration of 0.2-5 mM, such as 0.2-4 mM, 0.2-3 mM, 0.2-2 mM, 0.2-1 mM, 0.2-0.5 mM, 0.2-0.4 mM or 0.2-0.3 mM; or at a total concentration of 0.3-5 mM, such as 0.3-4 mM, 0.3-3 mM, 0.3-2 mM, 0.3-1 mM, 0.3-0.5 mM or 0.3-0.4 mM; or at a total concentration of 0.4-5 mM, such as 0.4-4 mM, 0.4-3 mM, 0.4-2 mM, 0.4-1 mM or 0.4-0.5 mM; or at a total concentration of 0.5-5 mM, such as 0.5-4 mM, 0.5-3 mM, 0.5-2 mM or 0.5-1 mM.

In one embodiment, there is provided an aqueous solution composition of pH in the range 4.0 to 6.0 comprising or consisting of:
terlipressin or a salt thereof;
optionally one or more buffers being substances having at least one ionisable group with a pKₐ in the range 3.0 to 7.0 and which pKₐ is within 1 pH unit of the pH of the composition;
an amino acid;
an uncharged tonicity modifier selected from the group consisting of sugars (such as sucrose, trehalose and lactose), sugar alcohols (such as mannitol and sorbitol), other polyols (such as glycerol and 1,2-propanediol) and polyethylene glycols (such as PEG300 and PEG400);
optionally one or more preservatives; and
optionally one or more non-ionic surfactants;
   wherein the buffers are present in the composition at a total concentration of 0-5 mM, such as 0-4 mM, 0-3 mM, 0-2 mM, 0-1 mM, 0-0.5 mM, 0-0.4 mM, 0-0.3 mM, 0-0.2 mM or 0-0.1 mM; or at a total concentration of 0.1-5 mM, such as 0.1-4 mM, 0.1-3 mM, 0.1-2 mM, 0.1-1 mM, 0.1-0.5 mM, 0.1-0.4 mM, 0.1-0.3 mM or 0.1-0.2 mM; or at a total concentration of 0.2-5 mM, such as 0.2-4 mM, 0.2-3 mM, 0.2-2 mM, 0.2-1 mM, 0.2-0.5 mM, 0.2-0.4 mM or 0.2-0.3 mM; or at a total concentration of 0.3-5 mM, such as 0.3-4 mM, 0.3-3 mM, 0.3-2 mM, 0.3-1 mM, 0.3-0.5 mM or 0.3-0.4 mM; or at a total concentration of 0.4-5 mM, such as 0.4-4 mM, 0.4-3 mM, 0.4-2 mM, 0.4-1 mM or 0.4-0.5 mM; or at a total concentration of 0.5-5 mM, such as 0.5-4 mM, 0.5-3 mM, 0.5-2 mM or 0.5-1 mM.

In one embodiment, there is provided an aqueous solution composition of pH in the range 4.0 to 6.0 comprising or consisting of:
terlipressin or a salt thereof;
optionally one or more buffers being substances having at least one ionisable group with a pKₐ in the range 3.0 to 7.0 and which pKₐ is within 1 pH unit of the pH of the composition;
an amino acid;
a charged tonicity modifier selected from sodium chloride and sodium sulfate;
optionally one or more preservatives; and
optionally one or more non-ionic surfactants;
   wherein the buffers are present in the composition at a total concentration of 0-5 mM, such as 0-4 mM, 0-3 mM, 0-2 mM, 0-1 mM, 0-0.5 mM, 0-0.4 mM, 0-0.3 mM, 0-0.2 mM or 0-0.1 mM; or at a total concentration of 0.1-5 mM, such as 0.1-4 mM, 0.1-3 mM, 0.1-2 mM, 0.1-1 mM, 0.1-0.5 mM, 0.1-0.4 mM, 0.1-0.3 mM or 0.1-0.2 mM; or at a total concentration of 0.2-5 mM, such as 0.2-4 mM, 0.2-3 mM, 0.2-2 mM, 0.2-1 mM, 0.2-0.5 mM, 0.2-0.4 mM or 0.2-0.3 mM; or at a total concentration of 0.3-5 mM, such as 0.3-4 mM, 0.3-3 mM, 0.3-2 mM, 0.3-1 mM, 0.3-0.5 mM or 0.3-0.4 mM; or at a total concentration of 0.4-5 mM, such as 0.4-4 mM, 0.4-3 mM, 0.4-2 mM, 0.4-1 mM or 0.4-0.5 mM; or at a total concentration of 0.5-5 mM, such as 0.5-4 mM, 0.5-3 mM, 0.5-2 mM or 0.5-1 mM.

In one embodiment, there is provided an aqueous solution composition of pH in the range 4.0 to 6.0 comprising or consisting of:
terlipressin or a salt thereof;
optionally one or more buffers being substances having at least one ionisable group with a pKₐ in the range 3.0 to 7.0 and which pKₐ is within 1 pH unit of the pH of the composition;
an amino acid selected from the group consisting of methionine, glycine, proline, arginine, lysine, aspartic acid, glutamic acid and histidine, in particular selected from the group consisting of methionine, glycine, proline, arginine and lysine, e.g. glycine, proline or methionine e.g. methionine;
an uncharged tonicity modifier selected from the group consisting of sugars (such as sucrose, trehalose and lactose), sugar alcohols (such as mannitol and sorbitol), other polyols (such as glycerol and 1,2-propanediol) and polyethylene glycols (such as PEG300 and PEG400);
optionally one or more preservatives; and
optionally one or more non-ionic surfactants;
   wherein the buffers are present in the composition at a total concentration of 0-5 mM, such as 0-4 mM, 0-3 mM, 0-2 mM, 0-1 mM, 0-0.5 mM, 0-0.4 mM, 0-0.3 mM, 0-0.2 mM or 0-0.1 mM; or at a total concentration of 0.1-5 mM, such as 0.1-4 mM, 0.1-3 mM, 0.1-2 mM, 0.1-1 mM, 0.1-0.5 mM, 0.1-0.4 mM, 0.1-0.3 mM or 0.1-0.2 mM; or at a total concentration of 0.2-5 mM, such as 0.2-4 mM, 0.2-3 mM, 0.2-2 mM, 0.2-1 mM, 0.2-0.5 mM, 0.2-0.4 mM or 0.2-0.3 mM; or at a total concentration of 0.3-5 mM, such as 0.3-4 mM, 0.3-3 mM, 0.3-2 mM, 0.3-1 mM, 0.3-0.5 mM or 0.3-0.4 mM; or at a total concentration of 0.4-5 mM, such as 0.4-4 mM, 0.4-3 mM, 0.4-2 mM, 0.4-1 mM or 0.4-0.5 mM; or at a total concentration of 0.5-5 mM, such as 0.5-4 mM, 0.5-3 mM, 0.5-2 mM or 0.5-1 mM.

In one embodiment, there is provided an aqueous solution composition of pH in the range 4.0 to 6.0 comprising or consisting of:
terlipressin or a salt thereof;
optionally one or more buffers being substances having at least one ionisable group with a pKₐ in the range 3.0 to 7.0 and which pKₐ is within 1 pH unit of the pH of the composition;
an amino acid selected from the group consisting of methionine, glycine, proline, arginine, lysine, aspartic acid, glutamic acid and histidine, in particular selected from the group consisting of methionine, glycine, proline, arginine and lysine, e.g. glycine, proline or methionine e.g. methionine;
a charged tonicity modifier selected from sodium chloride and sodium sulfate;
optionally one or more preservatives; and
optionally one or more non-ionic surfactants;
   wherein the buffers are present in the composition at a total concentration of 0-5 mM, such as 0-4 mM, 0-3 mM, 0-2 mM, 0-1 mM, 0-0.5 mM, 0-0.4 mM, 0-0.3 mM, 0-0.2 mM or 0-0.1 mM; or at a total concentration of 0.1-5 mM, such as 0.1-4 mM, 0.1-3 mM, 0.1-2 mM, 0.1-1 mM, 0.1-0.5 mM, 0.1-0.4 mM, 0.1-0.3 mM or 0.1-0.2 mM; or at a total concentration of 0.2-5 mM, such as 0.2-4 mM, 0.2-3 mM, 0.2-2 mM, 0.2-1 mM, 0.2-0.5 mM, 0.2-0.4 mM or 0.2-0.3 mM; or at a total concentration of 0.3-5 mM, such as 0.3-4 mM, 0.3-3 mM, 0.3-2 mM, 0.3-1 mM, 0.3-0.5 mM or 0.3-0.4 mM; or at a total concentration of 0.4-5 mM, such as 0.4-4 mM, 0.4-3 mM, 0.4-2 mM, 0.4-1 mM or 0.4-0.5 mM; or at a total concentration of 0.5-5 mM, such as 0.5-4 mM, 0.5-3 mM, 0.5-2 mM or 0.5-1 mM.

In one embodiment, there is provided an aqueous solution composition of pH in the range 4.0 to 6.0 e.g. around 5.0 comprising or consisting of:
terlipressin or a salt thereof e.g. terlipressin acetate;
a buffer selected from succinate and benzoate buffer, the buffer being present in the composition at a concentration of 0.5-5 mM e.g. 0.5-3 mM e.g. 0.5-1 mM;
optionally an amino acid selected from the group consisting of methionine, glycine, proline, arginine, lysine, aspartic acid, glutamic acid and histidine, in particular selected from the group consisting of methionine, glycine, proline, arginine and lysine, e.g. glycine, proline or methionine e.g. glycine;
a tonicity modifier e.g. sodium chloride e.g. at a concentration of 100-200 mM e.g. about 150 mM;
optionally one or more preservatives; and
optionally one or more non-ionic surfactants.

In one embodiment, there is provided an aqueous solution composition of pH in the range 4.0 to 6.0 e.g. around 4.5 or around 5.0 comprising or consisting of:
terlipressin or a salt thereof e.g. terlipressin acetate;
a buffer selected from acetate and lactate buffer (particularly acetate buffer);
wherein buffers are present in the composition at a concentration of 0.5-5 mM e.g. 0.5-3 mM e.g. 0.5-1 mM;
optionally an amino acid selected from the group consisting of methionine, glycine, proline, arginine, lysine, aspartic acid, glutamic acid and histidine, in particular selected from the group consisting of methionine, glycine, proline, arginine and lysine, e.g. glycine, proline or methionine;
a tonicity modifier e.g. sodium chloride e.g. at a concentration of 100-200 mM e.g. about 150 mM or e.g. a non-ionic tonicity modifier such as mannitol e.g. at a concentration of 50-1000 mM, such as 200-500 mM, or about 300 mM;
optionally one or more preservatives; and
optionally one or more non-ionic surfactants.

The presently claimed invention derives from the surprising observation that compositions comprising terlipressin are stabilized at low buffer concentrations. Such solutions may be further stabilized by the addition of an additive such as an amino acid and/or a tonicity modifier.

Suitably the composition of the invention remains as a clear solution following storage at 2-8° C. for an extended period of time, such as at least 6 months, preferably 12 months, most preferably at least 18 months.

Suitably the composition of the invention remains as a clear solution following storage at 25° C. for an extended period of time, such as at least 6 months, preferably at least 12 months, such as at least 18 months, such as at least 24 months.

Suitably the composition of the invention remains as a clear solution following storage at 30° C. for an extended period of time, such as at least 6 months, preferably at least 12 months, such as at least 18 months, such as at least 24 months.

Suitably the composition of the invention has improved storage stability either at 2-8° C. or at increased temperature than in an equivalent composition that comprises higher concentration of the same buffer or buffers.

Suitably the composition of the invention has improved storage stability either at 2-8° C. or at increased temperature than in an equivalent composition that does not comprise the amino acid and/or tonicity modifier.

In one embodiment, the composition of the invention comprises no more than 5% total impurities, such as no more than 4%, such as no more than 3%, such as no more than 2% total impurities (by total weight of terlipressin in the composition, as measured by RP-HPLC (Reversed-Phase High-Performance Liquid Chromatography) or a similar suitable technique) following storage at 2-8° C. for at least 6 months, preferably 12 months, most preferably 18 months.

In one embodiment, the composition of the invention comprises no more than 5% total impurities, such as no more than 4%, such as no more than 3%, such as no more than 2% total impurities (by total weight of terlipressin in the composition, as measured by RP-HPLC or a similar suitable technique) following storage at 25° C. for at least 6 months, preferably 12 months, such as 18 months or 24 months.

In one embodiment, the composition of the invention comprises no more than 5% total impurities, such as no more than 4%, such as no more than 3%, such as no more than 2% total impurities (by total weight of terlipressin in the composition, as measured by RP-HPLC or a similar suitable technique) following storage at 30° C. for at least 6 months, preferably 12 months, such as 18 months or 24 months.

In one embodiment, the composition of the invention comprises lower level of impurities (as measured by RP-HPLC or a similar suitable technique) than a commercially available composition comprising terlipressin (as measured by RP-HPLC or a similar suitable technique) following storage at 2-8° C. for at least 6 months, preferably 12 months, most preferably 18 months.

In one embodiment, the composition of the invention comprises lower level of impurities (as measured by RP-HPLC or a similar suitable technique) than a commercially available composition comprising terlipressin (as measured by RP-HPLC or a similar suitable technique) following storage at 25° C. for at least 6 months, preferably 12 months, such as 18 months or 24 months.

In one embodiment, the composition of the invention comprises lower level of impurities (as measured by RP-HPLC or a similar suitable technique) than a commercially available composition comprising terlipressin (as measured by RP-HPLC or a similar suitable technique) following storage at 30° C. for at least 6 months, preferably 12 months, such as 18 months or 24 months.

In a further aspect of the invention, there is provided a method of improving the stability of an aqueous solution composition of pH in the range of 4.0 to 6.0 comprising terlipressin, which comprises maintaining a low buffer concentration, wherein the buffer or buffers being substances having at least one ionisable group with a pKₐ in the range 3.0 to 7.0 and which pKₐ is within 1 pH unit of the pH of the composition are present in the composition at a concentration of 0-5 mM, such as 0-4 mM, 0-3 mM, 0-2 mM, 0-1 mM, 0-0.5 mM, 0-0.4 mM, 0-0.3 mM, 0-0.2 mM or 0-0.1 mM; or at a total concentration of 0.1-5 mM, such as 0.1-4 mM, 0.1-3 mM, 0.1-2 mM, 0.1-1 mM, 0.1-0.5 mM, 0.1-0.4 mM, 0.1-0.3 mM or 0.1-0.2 mM; or at a total concentration of 0.2-5 mM, such as 0.2-4 mM, 0.2-3 mM, 0.2-2 mM, 0.2-1 mM, 0.2-0.5 mM, 0.2-0.4 mM or 0.2-0.3 mM; or at a total concentration of 0.3-5 mM, such as 0.3-4 mM, 0.3-3 mM, 0.3-2 mM, 0.3-1 mM, 0.3-0.5 mM or 0.3-0.4 mM; or at a total concentration of 0.4-5 mM, such as 0.4-4 mM, 0.4-3 mM, 0.4-2 mM, 0.4-1 mM or 0.4-0.5 mM; or at a total concentration of 0.5-5 mM, such as 0.5-4 mM, 0.5-3 mM, 0.5-2 mM or 0.5-1 mM.

In a further aspect of the invention, there is provided a method of improving the stability of an aqueous solution composition of pH in the range of 4.0 to 6.0 comprising terlipressin or a salt thereof, which comprises:
i. maintaining a low buffer concentration, wherein the buffer or buffers being substances having at least one ionisable group with a pKₐ in the range 3.0 to 7.0 and which pKₐ is within 1 pH unit of the pH of the composition are present in the composition at a concentration of 0-5 mM, such as 0-4 mM, 0-3 mM, 0-2 mM, 0-1 mM, 0-0.5 mM, 0-0.4 mM, 0-0.3 mM, 0-0.2 mM or 0-0.1 mM; or at a total concentration of 0.1-5 mM, such as 0.1-4 mM, 0.1-3 mM, 0.1-2 mM, 0.1-1 mM, 0.1-0.5 mM, 0.1-0.4 mM, 0.1-0.3 mM or 0.1-0.2 mM; or at a total concentration of 0.2-5 mM, such as 0.2-4 mM, 0.2-3 mM, 0.2-2 mM, 0.2-1 mM, 0.2-0.5 mM, 0.2-0.4 mM or 0.2-0.3 mM; or at a total concentration of 0.3-5 mM, such as 0.3-4 mM, 0.3-3 mM, 0.3-2 mM, 0.3-1 mM, 0.3-0.5 mM or 0.3-0.4 mM; or at a total concentration of 0.4-5 mM, such as 0.4-4 mM, 0.4-3 mM, 0.4-2 mM, 0.4-1 mM or 0.4-0.5 mM; or at a total concentration of 0.5-5 mM, such as 0.5-4 mM, 0.5-3 mM, 0.5-2 mM or 0.5-1 mM; and
ii. adding an amino acid, suitably selected from the group consisting of methionine, glycine, proline, arginine, lysine, aspartic acid, glutamic acid and histidine, in particular selected from the group consisting of methionine, glycine, proline, arginine and lysine, e.g. glycine, proline or methionine, e.g. methionine.

In a further aspect of the invention, there is provided a method of improving the stability of an aqueous solution composition of pH in the range of 4.0 to 6.0 comprising terlipressin or a salt thereof, which comprises:
i. maintaining a low buffer concentration, wherein the buffer or buffers being substances having at least one ionisable group with a pKₐ in the range 3.0 to 7.0 and which pKₐ is within 1 pH unit of the pH of the composition are present in the composition at a concentration of 0-5 mM, such as 0-4 mM, 0-3 mM, 0-2 mM, 0-1 mM, 0-0.5 mM, 0-0.4 mM, 0-0.3 mM, 0-0.2 mM or 0-0.1 mM; or at a total concentration of 0.1-5 mM, such as 0.1-4 mM, 0.1-3 mM, 0.1-2 mM, 0.1-1 mM, 0.1-0.5 mM, 0.1-0.4 mM, 0.1-0.3 mM or 0.1-0.2 mM; or at a total concentration of 0.2-5 mM, such as 0.2-4 mM, 0.2-3 mM, 0.2-2 mM, 0.2-1 mM, 0.2-0.5 mM, 0.2-0.4 mM or 0.2-0.3 mM; or at a total concentration of 0.3-5 mM, such as 0.3-4 mM, 0.3-3 mM, 0.3-2 mM, 0.3-1 mM, 0.3-0.5 mM or 0.3-0.4 mM; or at a total concentration of 0.4-5 mM, such as 0.4-4 mM, 0.4-3 mM, 0.4-2 mM, 0.4-1 mM or 0.4-0.5 mM; or at a total concentration of 0.5-5 mM, such as 0.5-4 mM, 0.5-3 mM, 0.5-2 mM or 0.5-1 mM; and
ii. adding an uncharged tonicity modifier, suitably selected from the group consisting of sugars (such as sucrose, trehalose and lactose), sugar alcohols (such as mannitol and sorbitol), other polyols (such as glycerol and 1,2-propanediol) and polyethylene glycols (such as PEG300 and PEG400).

In a further aspect of the invention, there is provided a method of improving the stability of an aqueous solution composition of pH in the range of 4.0 to 6.0 comprising terlipressin or a salt thereof, which comprises:
i. maintaining a low buffer concentration, wherein the buffer or buffers substances having at least one ionisable group with a pKₐ in the range 3.0 to 7.0 and which pKₐ is within 1 pH unit of the pH of the composition are present in the composition at a concentration of 0-5 mM, such as 0-4 mM, 0-3 mM, 0-2 mM, 0-1 mM, 0-0.5 mM, 0-0.4 mM, 0-0.3 mM, 0-0.2 mM or 0-0.1 mM; or at a total concentration of 0.1-5 mM, such as 0.1-4 mM, 0.1-3 mM, 0.1-2 mM, 0.1-1 mM, 0.1-0.5 mM, 0.1-0.4 mM, 0.1-0.3 mM or 0.1-0.2 mM; or at a total concentration of 0.2-5 mM, such as 0.2-4 mM, 0.2-3 mM, 0.2-2 mM, 0.2-1 mM, 0.2-0.5 mM, 0.2-0.4 mM or 0.2-0.3 mM; or at a total concentration of 0.3-5 mM, such as 0.3-4 mM, 0.3-3 mM, 0.3-2 mM, 0.3-1 mM, 0.3-0.5 mM or 0.3-0.4 mM; or at a total concentration of 0.4-5 mM, such as 0.4-4 mM, 0.4-3 mM, 0.4-2 mM, 0.4-1 mM or 0.4-0.5 mM; or at a total concentration of 0.5-5 mM, such as 0.5-4 mM, 0.5-3 mM, 0.5-2 mM or 0.5-1 mM; and
ii. adding a charged tonicity modifier, suitably selected from sodium chloride and sodium sulfate.

In a further aspect of the invention, there is provided a method of improving the stability of an aqueous solution composition of pH in the range of 4.0 to 6.0 comprising terlipressin or a salt thereof, which comprises:
i. maintaining a low buffer concentration, wherein the buffer or buffers being substances having at least one ionisable group with a pKₐ in the range 3.0 to 7.0 and which pKₐ is within 1 pH unit of the pH of the composition are present in the composition at a concentration of 0-5 mM, such as 0-4 mM, 0-3 mM, 0-2 mM, 0-1 mM, 0-0.5 mM, 0-0.4 mM, 0-0.3 mM, 0-0.2 mM or 0-0.1 mM; or at a total concentration of 0.1-5 mM, such as 0.1-4 mM, 0.1-3 mM, 0.1-2 mM, 0.1-1 mM, 0.1-0.5 mM, 0.1-0.4 mM, 0.1-0.3 mM or 0.1-0.2 mM; or at a total concentration of 0.2-5 mM, such as 0.2-4 mM, 0.2-3 mM, 0.2-2 mM, 0.2-1 mM, 0.2-0.5 mM, 0.2-0.4 mM or 0.2-0.3 mM; or at a total concentration of 0.3-5 mM, such as 0.3-4 mM, 0.3-3 mM, 0.3-2 mM, 0.3-1 mM, 0.3-0.5 mM or 0.3-0.4 mM; or at a total concentration of 0.4-5 mM, such as 0.4-4 mM, 0.4-3 mM, 0.4-2 mM, 0.4-1 mM or 0.4-0.5 mM; or at a total concentration of 0.5-5 mM, such as 0.5-4 mM, 0.5-3 mM, 0.5-2 mM or 0.5-1 mM;
ii. adding an amino acid selected from the group consisting of methionine, glycine, proline, arginine, lysine, aspartic acid, glutamic acid and histidine, in particular selected from the group consisting of methionine, glycine, proline, arginine and lysine, e.g. glycine, proline or methionine, e.g. methionine; and
ii. adding an uncharged tonicity modifier, suitably selected the group consisting of sugars (such as sucrose, trehalose and lactose), sugar alcohols (such as mannitol and sorbitol), other polyols (such as glycerol and 1,2-propanediol) and polyethylene glycols (such as PEG300 and PEG400).

In a further aspect of the invention, there is provided a method of improving the stability of an aqueous solution composition of pH in the range of 4.0 to 6.0 comprising terlipressin or a salt thereof, which comprises:
i. maintaining a low buffer concentration, wherein the buffer or buffers substances having at least one ionisable group with a pKₐ in the range 3.0 to 7.0 and which pKₐ is within 1 pH unit of the pH of the composition are present in the composition at a concentration of 0-5 mM, such as 0-4 mM, 0-3 mM, 0-2 mM, 0-1 mM, 0-0.5 mM, 0-0.4 mM, 0-0.3 mM, 0-0.2 mM or 0-0.1 mM; or at a total concentration of 0.1-5 mM, such as 0.1-4 mM, 0.1-3 mM, 0.1-2 mM, 0.1-1 mM, 0.1-0.5 mM, 0.1-0.4 mM, 0.1-0.3 mM or 0.1-0.2 mM; or at a total concentration of 0.2-5 mM, such as 0.2-4 mM, 0.2-3 mM, 0.2-2 mM, 0.2-1 mM, 0.2-0.5 mM, 0.2-0.4 mM or 0.2-0.3 mM; or at a total concentration of 0.3-5 mM, such as 0.3-4 mM, 0.3-3 mM, 0.3-2 mM, 0.3-1 mM, 0.3-0.5 mM or 0.3-0.4 mM; or at a total concentration of 0.4-5 mM, such as 0.4-4 mM, 0.4-3 mM, 0.4-2 mM, 0.4-1 mM or 0.4-0.5 mM; or at a total concentration of 0.5-5 mM, such as 0.5-4 mM, 0.5-3 mM, 0.5-2 mM or 0.5-1 mM;
ii. adding an amino acid selected from the group consisting of methionine, glycine, proline, arginine, lysine, aspartic acid, glutamic acid and histidine, in particular selected from the group consisting of methionine, glycine, proline, arginine and lysine, e.g. glycine, proline or methionine, e.g. methionine; and
ii. adding a charged tonicity modifier, suitably selected from sodium chloride and sodium sulfate.

In a further aspect of the invention, there is provided the use of an amino acid (suitably selected from the group consisting of methionine, glycine, proline, arginine, lysine, aspartic acid, glutamic acid and histidine, in particular selected from the group consisting of methionine, glycine, proline, arginine and lysine, e.g. glycine, proline or methionine, e.g. methionine) for improving the stability of an aqueous solution composition of pH in the range of 4.0 to 6.0 comprising terlipressin or a salt thereof, wherein the composition optionally comprises buffer or buffers being substances having at least one ionisable group with a pKₐ in the range 3.0 to 7.0 and which pKₐ is within 1 pH unit of the pH of the composition, wherein the buffer or buffers are present in the composition at a total concentration of 0-5 mM, such as 0-4 mM, 0-3 mM, 0-2 mM, 0-1 mM, 0-0.5 mM, 0-0.4 mM, 0-0.3 mM, 0-0.2 mM or 0-0.1 mM; or at a total concentration of 0.1-5 mM, such as 0.1-4 mM, 0.1-3 mM, 0.1-2 mM, 0.1-1 mM, 0.1-0.5 mM, 0.1-0.4 mM, 0.1-0.3 mM or 0.1-0.2 mM; or at a total concentration of 0.2-5 mM, such as 0.2-4 mM, 0.2-3 mM, 0.2-2 mM, 0.2-1 mM, 0.2-0.5 mM, 0.2-0.4 mM or 0.2-0.3 mM; or at a total concentration of 0.3-5 mM, such as 0.3-4 mM, 0.3-3 mM, 0.3-2 mM, 0.3-1 mM, 0.3-0.5 mM or 0.3-0.4 mM; or at a total concentration of 0.4-5 mM, such as 0.4-4 mM, 0.4-3 mM, 0.4-2 mM, 0.4-1 mM or 0.4-0.5 mM; or at a total concentration of 0.5-5 mM, such as 0.5-4 mM, 0.5-3 mM, 0.5-2 mM or 0.5-1 mM.

In a further aspect of the invention, there is provided the use of an uncharged tonicity modifier (suitably selected from the group consisting of sugars (such as sucrose, trehalose and lactose), sugar alcohols (such as mannitol and sorbitol), other polyols (such as glycerol and 1,2-propanediol) and polyethylene glycols (such as PEG300 and PEG400)) for improving the stability of an aqueous solution composition of pH in the range of 4.0 to 6.0 comprising terlipressin or a salt thereof, wherein the composition optionally comprises buffer or buffers being substances having at least one ionisable group with a pKₐ in the range 3.0 to 7.0 and which pKₐ is within 1 pH unit of the pH of the composition, wherein the buffer or buffers are present in the composition at a total concentration of 0-5 mM, such as 0-4 mM, 0-3 mM, 0-2 mM, 0-1 mM, 0-0.5 mM, 0-0.4 mM, 0-0.3 mM, 0-0.2 mM or 0-0.1 mM; or at a total concentration of 0.1-5 mM, such as 0.1-4 mM, 0.1-3 mM, 0.1-2 mM, 0.1-1 mM, 0.1-0.5 mM, 0.1-0.4 mM, 0.1-0.3 mM or 0.1-0.2 mM; or at a total concentration of 0.2-5 mM, such as 0.2-4 mM, 0.2-3 mM, 0.2-2 mM, 0.2-1 mM, 0.2-0.5 mM, 0.2-0.4 mM or 0.2-0.3 mM; or at a total concentration of 0.3-5 mM, such as 0.3-4 mM, 0.3-3 mM, 0.3-2 mM, 0.3-1 mM, 0.3-0.5 mM or 0.3-0.4 mM; or at a total concentration of 0.4-5 mM, such as 0.4-4 mM, 0.4-3 mM, 0.4-2 mM, 0.4-1 mM or 0.4-0.5 mM; or at a total concentration of 0.5-5 mM, such as 0.5-4 mM, 0.5-3 mM, 0.5-2 mM or 0.5-1 mM.

In a further aspect of the invention, there is provided the use of a charged tonicity modifier (suitably selected from sodium chloride and sodium sulphate) for improving the stability of an aqueous solution composition of pH in the range of 4.0 to 6.0 comprising terlipressin or a salt thereof, wherein the composition optionally comprises buffer or buffers being substances having at least one ionisable group with a pKₐ in the range 3.0 to 7.0 and which pKₐ is within 1 pH unit of the pH of the composition, wherein the buffer or buffers are present in the composition at a total concentration of 0-5 mM, such as 0-4 mM, 0-3 mM, 0-2 mM, 0-1 mM, 0-0.5 mM, 0-0.4 mM, 0-0.3 mM, 0-0.2 mM or 0-0.1 mM; or at a total concentration of 0.1-5 mM, such as 0.1-4 mM, 0.1-3 mM, 0.1-2 mM, 0.1-1 mM, 0.1-0.5 mM, 0.1-0.4 mM, 0.1-0.3 mM or 0.1-0.2 mM; or at a total concentration of 0.2-5 mM, such as 0.2-4 mM, 0.2-3 mM, 0.2-2 mM, 0.2-1 mM, 0.2-0.5 mM, 0.2-0.4 mM or 0.2-0.3 mM; or at a total concentration of 0.3-5 mM, such as 0.3-4 mM, 0.3-3 mM, 0.3-2 mM, 0.3-1 mM, 0.3-0.5 mM or 0.3-0.4 mM; or at a total concentration of 0.4-5 mM, such as 0.4-4 mM, 0.4-3 mM, 0.4-2 mM, 0.4-1 mM or 0.4-0.5 mM; or at a total concentration of 0.5-5 mM, such as 0.5-4 mM, 0.5-3 mM, 0.5-2 mM or 0.5-1 mM.

In a further aspect of the invention, there is provided the use of a minimal amount of buffer for improving the stability of an aqueous solution composition of pH in the range of 4.0 to 6.0 comprising terlipressin or a salt thereof, wherein the composition comprises an amino acid (suitably selected from the group consisting of methionine, glycine, proline, arginine, lysine, aspartic acid, glutamic acid and histidine, in particular selected from the group consisting of methionine, glycine, proline, arginine and lysine, e.g. glycine, proline or methionine, e.g. methionine), and wherein the composition optionally comprises buffer or buffers being substances having at least one ionisable group with a pKₐ in the range 3.0 to 7.0 and which pKₐ is within 1 pH unit of the pH of the composition, wherein the buffer or buffers are present in the composition at a total concentration of 0-5 mM, such as 0-4 mM, 0-3 mM, 0-2 mM, 0-1 mM, 0-0.5 mM, 0-0.4 mM, 0-0.3 mM, 0-0.2 mM or 0-0.1 mM; or at a total concentration of 0.1-5 mM, such as 0.1-4 mM, 0.1-3 mM, 0.1-2 mM, 0.1-1 mM, 0.1-0.5 mM, 0.1-0.4 mM, 0.1-0.3 mM or 0.1-0.2 mM; or at a total concentration of 0.2-5 mM, such as 0.2-4 mM, 0.2-3 mM, 0.2-2 mM, 0.2-1 mM, 0.2-0.5 mM, 0.2-0.4 mM or 0.2-0.3 mM; or at a total concentration of 0.3-5 mM, such as 0.3-4 mM, 0.3-3 mM, 0.3-2 mM, 0.3-1 mM, 0.3-0.5 mM or 0.3-0.4 mM; or at a total concentration of 0.4-5 mM, such as 0.4-4 mM, 0.4-3 mM, 0.4-2 mM, 0.4-1 mM or 0.4-0.5 mM; or at a total concentration of 0.5-5 mM, such as 0.5-4 mM, 0.5-3 mM, 0.5-2 mM or 0.5-1 mM.

In an embodiment, the composition of the invention is a composition for use in therapy. In an embodiment, the composition of the invention is a pharmaceutical composition.

Terlipressin is indicated inter alia for the treatment of hypotension, septic shock, esophageal varices and hepatorenal syndrome.

Accordingly, the invention provides a composition of the invention for use in the treatment of hypotension, septic shock, esophageal varices or hepatorenal syndrome. It also provides use of a composition of the invention for the manufacture of a medicament for the treatment of hypotension, septic shock, esophageal varices or hepatorenal syndrome. It also provides a method of treatment of hypotension, septic shock, esophageal varices or hepatorenal syndrome which comprises administering to a patient, particularly a human patient, in need thereof a therapeutically effective amount of a composition of the invention.

In one embodiment is provided an aqueous solution composition of pH in the range 4.0-6.0 for use in therapy comprising:
terlipressin or a salt thereof;
optionally one or more buffers being substances having at least one ionisable group with a pKₐ in the range 3.0 to 7.0 and which pKₐ is within 1 pH unit of the pH of the composition;
optionally an amino acid; and
optionally a tonicity modifier;
   wherein the buffers are present in the composition at a total concentration of 0-5 mM, such as 0-4 mM, 0-3 mM, 0-2 mM, 0-1 mM, 0-0.5 mM, 0-0.4 mM, 0-0.3 mM, 0-0.2 mM or 0-0.1 mM; or at a total concentration of 0.1-5 mM, such as 0.1-4 mM, 0.1-3 mM, 0.1-2 mM, 0.1-1 mM, 0.1-0.5 mM, 0.1-0.4 mM, 0.1-0.3 mM or 0.1-0.2 mM; or at a total concentration of 0.2-5 mM, such as 0.2-4 mM, 0.2-3 mM, 0.2-2 mM, 0.2-1 mM, 0.2-0.5 mM, 0.2-0.4 mM or 0.2-0.3 mM; or at a total concentration of 0.3-5 mM, such as 0.3-4 mM, 0.3-3 mM, 0.3-2 mM, 0.3-1 mM, 0.3-0.5 mM or 0.3-0.4 mM; or at a total concentration of 0.4-5 mM, such as 0.4-4 mM, 0.4-3 mM, 0.4-2 mM, 0.4-1 mM or 0.4-0.5 mM; or at a total concentration of 0.5-5 mM, such as 0.5-4 mM, 0.5-3 mM, 0.5-2 mM or 0.5-1 mM.

In one embodiment is provided an aqueous solution composition which is a pharmaceutical composition, of pH in the range 4.0-6.0 comprising:
terlipressin or a salt thereof;
optionally one or more buffers being substances having at least one ionisable group with a pKₐ in the range 3.0 to 7.0 and which pKₐ is within 1 pH unit of the pH of the composition;
optionally an amino acid; and
optionally a tonicity modifier;
   wherein the buffers are present in the composition at a total concentration of 0-5 mM, such as 0-4 mM, 0-3 mM, 0-2 mM, 0-1 mM, 0-0.5 mM, 0-0.4 mM, 0-0.3 mM, 0-0.2 mM or 0-0.1 mM; or at a total concentration of 0.1-5 mM, such as 0.1-4 mM, 0.1-3 mM, 0.1-2 mM, 0.1-1 mM, 0.1-0.5 mM, 0.1-0.4 mM, 0.1-0.3 mM or 0.1-0.2 mM; or at a total concentration of 0.2-5 mM, such as 0.2-4 mM, 0.2-3 mM, 0.2-2 mM, 0.2-1 mM, 0.2-0.5 mM, 0.2-0.4 mM or 0.2-0.3 mM; or at a total concentration of 0.3-5 mM, such as 0.3-4 mM, 0.3-3 mM, 0.3-2 mM, 0.3-1 mM, 0.3-0.5 mM or 0.3-0.4 mM; or at a total concentration of 0.4-5 mM, such as 0.4-4 mM, 0.4-3 mM, 0.4-2 mM, 0.4-1 mM or 0.4-0.5 mM; or at a total concentration of 0.5-5 mM, such as 0.5-4 mM, 0.5-3 mM, 0.5-2 mM or 0.5-1 mM.

All embodiments described above with respect to the aqueous solution composition apply equally to methods and uses of the invention.

There is also provided a container, for example made of plastics or glass, containing one dose or a plurality of doses of the composition as described herein. The container can be for example, a vial, a pre-filled syringe, a pre-filled infusion bag, or a cartridge designed to be a replaceable item for use with an injection device.

The compositions of the invention may suitably be packaged for injection, especially intravenous infusion, intravenous injection, subcutaneous injection or intramuscular injection.

An aspect of the invention is an injection device, particularly a device adapted for subcutaneous or intramuscular injection, for single or multiple use comprising a container containing one dose or a plurality of doses of the composition of the invention together with an injection needle. In an embodiment, the container is a replaceable cartridge which contains a plurality of doses. In an embodiment, the needle is replaceable e.g. after each occasion of use. In one embodiment, the injection device is in the form of a pen.

Compositions according to the invention are expected to have good physical and chemical stability as described herein.

### Additional Clauses of the Invention

1. An aqueous solution composition of pH in the range 4.0-6.0 comprising:
terlipressin or a salt thereof;
optionally one or more buffers being substances having at least one ionisable group with a pKₐ in the range 3.0 to 7.0 and which pKₐ is within 1 pH unit of the pH of the composition;
optionally an amino acid; and
optionally a tonicity modifier
   wherein the buffers are present in the composition at a total concentration of 0-5 mM.
   2. An aqueous solution composition according to clause 1, wherein the concentration of terlipressin or salt thereof in the composition is 0.001-50 mg/ml, such as 0.01-10 mg/ml, 0.01-5 mg/ml, 0.01-3 mg/ml, 0.01-2 mg/ml, 0.01-1 mg/ml or 0.1-1 mg/ml.
   3. An aqueous solution composition according to clause 1 or clause 2, wherein the total concentration of buffers in the composition is 0.1-5 mM, such as 0.1-4 mM, 0.1-3 mM, 0.1-2 mM, 0.1-1 mM, 0.1-0.5 mM, 0.1-0.4 mM, 0.1-0.3 mM or 0.1-0.2 mM.
   4. An aqueous solution composition according to any one of clauses 1 to 3, wherein the total concentration of buffers in the composition is ≤5 mM, such as ≤4 mM, ≤3 mM, ≤2 mM, ≤1 mM, ≤0.5 mM, ≤0.4 mM, ≤0.3 mM, ≤0.2 mM or ≤0.1 mM.
   5. An aqueous solution composition according to any one of clauses 1 to 4, wherein the aqueous solution composition is substantially free of buffer.
   6. An aqueous solution composition according to any one of clauses 1 to 5, wherein the buffer or buffers is/are selected from the group consisting of histidine, maleate, glyoxylate, aspartame, glucuronate, aspartate, glutamate, tartrate, gluconate, lactate, glycolic acid, adenine, succinate, ascorbate, benzoate, phenylacetate, gallate, cytosine, p-aminobenzoic acid, sorbate, acetate, propionate, alginate, urate, 2-(N-morpholino)ethanesulphonic acid, bicarbonate, bis(2-hydroxyethyl) iminotris(hydroxymethyl)methane, N-(2-acetamido)-2-iminodiacetic acid, 2-[(2-amino-2-oxoethyl)amino]ethanesulphonic acid and piperazine, N,N′-bis(2-ethanesulphonic acid) and salts thereof, and combinations thereof.
   7. An aqueous solution composition according to clause 6, wherein the buffer is selected from the group consisting of histidine, maleate, tartrate, lactate, benzoate, acetate and bicarbonate, particularly lactate and acetate especially acetate.
   8. An aqueous solution composition according to clause 6, wherein the buffer is or comprises a buffer selected from the group consisting of benzoate and succinate buffer.
   9. An aqueous solution composition according to any one of clauses 1 to 8, wherein an amino acid is present.
   10. An aqueous solution composition according to clause 9, wherein an amino acid is present and is selected from the group consisting of glycine, proline, methionine, arginine, lysine, aspartic acid, glutamic acid and histidine.
   11. An aqueous solution composition according to clause 10, wherein an amino acid is selected from the group consisting of glycine, proline, methionine, arginine and lysine, and in particular is glycine, proline or methionine, e.g. methionine.
   12. An aqueous solution composition according to clause 10, wherein the amino acid is glycine.
   13. An aqueous solution composition according to any one of clauses 9 to 12, wherein an amino acid is present at a concentration of 1-200 mM, such as 1-100 mM, 1-50 mM, 1-20 mM, 1-10 mM, 1-5 mM, 1-4 mM, 1-3 mM or 1-2 mM.
   14. An aqueous solution composition of pH in the range 4.0-6.0 comprising:
terlipressin or a salt thereof;
one or more buffers being substances having at least one ionisable group with a pKₐ in the range 3.0 to 7.0 and which pKₐ is within 1 pH unit of the pH of the composition; and
an amino acid which does not comprise ionisable groups with pKₐ within 1 pH unit of the pH of the composition e.g. selected from methionine, glycine and proline;
   wherein the buffers are present in the composition at a total concentration of 0.5 to 5 mM e.g. 0.5 to 3 mM.
   15. An aqueous solution composition according to clause 14 wherein the amino acid is present at a concentration of 2-20 mM.
   16. An aqueous solution composition according to clause 14 or clause 15, wherein the buffer is selected from the group consisting of histidine, maleate, tartrate, lactate, benzoate, acetate and bicarbonate, particularly lactate and acetate especially acetate.
   17. An aqueous solution composition according to any one of clauses 1 to 16, wherein a tonicity modifier is present.
   18. An aqueous solution composition according to clause 17, wherein a tonicity modifier is present and is an uncharged tonicity modifier, suitably selected from the group consisting of glycerol, 1,2-propanediol, mannitol, sorbitol, sucrose, trehalose, lactose, PEG300 and PEG400, and in particular is selected from the group consisting of mannitol and sorbitol.
   19. An aqueous solution composition according to clause 17 or clause 18, wherein an uncharged tonicity modifier is present at a concentration of 50-1000 mM, such as 200-500 mM, or about 300 mM.
   20. An aqueous solution composition according to clause 17, wherein a tonicity modifier present and is a charged tonicity modifier.
   21. An aqueous solution composition according to clause 20, wherein a charged tonicity modifier is selected from the group consisting of sodium chloride and sodium sulphate.
   22. An aqueous solution composition according to clause 21, wherein a charged tonicity modifier is sodium chloride.
   23. An aqueous solution composition according to any one of clauses 20 to 22, wherein a charged tonicity modifier is present at a concentration of 25-500 mM, such as 50-250 mM, or about 150 mM.
   24. An aqueous solution composition according to any one of clauses 1 to 23, further comprising a non-ionic surfactant.
   25. An aqueous solution composition according to clause 24, wherein the non-ionic surfactant is selected from the group consisting of an alkyl glycoside, a polysorbate, an alkyl ether of polyethylene glycol, a block copolymer of polyethylene glycol and polypropylene glycol, and an alkylphenyl ether of polyethylene glycol.
   26. An aqueous solution composition according to clause 25, wherein the non-ionic surfactant is a polysorbate such as polysorbate 20 or polysorbate 80.
   27. An aqueous solution composition according to any one of clauses 24 to 26, wherein the non-ionic surfactant is present at a concentration of 10-2000 μg/ml, such as 50-1000 μg/ml, 100-500 μg/ml or about 200 μg/ml.
   28. An aqueous solution composition according to any one of clauses 1 to 27, which additionally comprises a preservative such as a phenolic or benzylic preservative.
   29. An aqueous solution composition according to clause 28, wherein the phenolic or benzylic preservative is selected from the group consisting of phenol, m-cresol, chlorocresol, benzyl alcohol, propyl paraben and methyl paraben.
   30. An aqueous solution composition according to clause 28 or clause 29, wherein the preservative is present at a concentration of 10-100 mM, such as 20-80 mM or 25-50 mM.
   31. An aqueous solution composition according to any one of clauses 1 to 30, wherein terlipressin is employed as terlipressin acetate.
   32. An aqueous solution composition according to any one of clauses 1 to 31, which comprises no more than 5% total impurities, such as no more than 4%, such as no more than 3%, such as no more than 2% total impurities (by total weight of terlipressin in the composition, as measured by RP-HPLC (Reversed-Phase High-Performance Liquid Chromatography) or a similar suitable technique) following storage at 2-8° C. for at least 6 months, preferably 12 months, most preferably 18 months.
   33. An aqueous solution composition according to any one of clauses 1 to 32, which comprises no more than 5% total impurities, such as no more than 4%, such as no more than 3%, such as no more than 2% total impurities (by total weight of terlipressin in the composition, as measured by RP-HPLC or a similar suitable technique) following storage at 25° C. for at least 6 months, preferably 12 months, such as 18 months or 24 months.
   34. An aqueous solution composition according to any one of clauses 1 to 33, which comprises no more than 5% total impurities, such as no more than 4%, such as no more than 3%, such as no more than 2% total impurities (by total weight of terlipressin in the composition, as measured by RP-HPLC or a similar suitable technique) following storage at 30° C. for at least 6 months, preferably 12 months, such as 18 months or 24 months.
   35. An aqueous solution composition according to any one of clauses 1 to 34, which is a pharmaceutical composition.
   36. An aqueous solution composition according to any one of clauses 1 to 35, which is a composition for use in therapy.
   37. An aqueous solution composition according to any one of clauses 1 to 35 for use in the treatment of hypotension, septic shock, esophageal varices or hepatorenal syndrome.
   38. Use of an aqueous solution composition according to any one of clauses 1 to 35 for the manufacture of a medicament for the treatment of hypotension, septic shock, esophageal varices or hepatorenal syndrome.
   39. A method of treatment of hypotension, septic shock, esophageal varices or hepatorenal syndrome which comprises administering to a patient, particularly a human patient, in need thereof a therapeutically effective amount of an aqueous solution composition according to any one of clauses 1 to 35.

### EXAMPLES

### General Methods

(a) Reversed-Phase Chromatography (RP-HPLC)

High performance reverse phase chromatography was performed using the Waters ACQUITY H-class Bio UPLC® system with a 1.7 μm Ethylene Bridged Hybrid particle, 130 Å pore resin trifunctionally immobilised with a C18 ligand in a 50 mm by 15 2.1 mm column. Mobile Phase A was 0.1 M Na₃PO₄ adjusted to pH 3.0 using trifluoroacetic acid. Mobile Phase B was prepared by mixing 2 parts (v/v) of acetonitrile with 1 part (v/v) of Mobile Phase A. The sample comprising a formulated peptide was bound in Mobile Phase A and eluted using a gradient of Mobile Phase A and Mobile Phase B. The sample volume was 10 μl, the flow rate was 0.4 mL/min, with 214 nm UV detection. All analyses were performed at 60° C.

(b) Visual Assessment

Visible particles are suitably detected using the 2.9.20. European Pharmacopoeia Monograph (Particulate Contamination: Visible Particles). The apparatus required consists of a viewing station comprising:
a matt black panel of appropriate size held in a vertical position
a non-glare white panel of appropriate size held in a vertical position next to the black panel
an adjustable lampholder fitted with a suitable, shaded, white-light source and with a suitable light diffuser (a viewing illuminator containing two 13 W fluorescent tubes, each 525 mm in length, is suitable). The intensity of illumination at the viewing point is maintained between 2000 lux and 3750 lux.

Any adherent labels are removed from the container and the outside washed and dried. The container is gently swirled or inverted, ensuring that air bubbles are not introduced, and observed for about 5 s in front of the white panel. The procedure is repeated in front of the black panel. The presence of any particles is recorded.

The visual scores are ranked as follows:

### Visual Assessment Scoring Method

Visual score 1: Clear solution, virtually free of particles
Visual score 2: ˜5 very small particles
Visual score 3: ˜10-20 very small particles
Visual score 4: 20-50 particles, including larger particles
Visual score 5: >50 particles, including larger particles

Whilst the particles in samples with visual scores 4 and 5 are clearly detectable on casual visual assessment under normal light, samples with visual score 1-3 generally appear as clear solutions on the same assessment. Samples with visual scores 1-3 are considered to be “Pass”; samples with visual score 4-5 are considered to be “Fail”.

### FORMULATION EXAMPLES

The following example formulations may be prepared:

Example A:

| | | |
|---|---|---|
| Terlipressin acetate | 100 | μg/ml |
| Sodium succinate | 1 | mM |
| Sodium chloride | 150 | mM |

| | |
|---|---|
| Water | qs |
| pH adjusted to 5.0 | |

Example B:

| Terlipressin acetate | 100 | μg/ml |
|---|---|---|
| Sodium succinate | 3 | mM |
| Sodium chloride | 150 | mM |
| Water for injection | qs | |
| pH adjusted to 5.0 | | |

Example C:

| Terlipressin acetate | 100 | μg/ml |
|---|---|---|
| Sodium succinate | 4.5 | mM |
| Sodium chloride | 150 | mM |
| Water for injection | qs | |
| pH adjusted to 5.0 | | |

Example D:

| Terlipressin acetate | 100 | μg/ml |
|---|---|---|
| Sodium succinate | 3 | mM |
| Sodium chloride | 140 | mM |
| Glycine | 10 | mM |
| Water for injection | qs | |
| pH adjusted to 5.0 | | |

Example E:

| Terlipressin acetate | 100 | μg/ml |
|---|---|---|
| Sodium benzoate | 1 | mM |
| Sodium chloride | 150 | mM |
| Water for injection | qs | |
| pH adjusted to 5.0 | | |

Example F:

| | | |
|---|---|---|
| Terlipressin acetate | 100 | μg/ml |
| Sodium benzoate | 3 | mM |
| Sodium chloride | 150 | mM |

| | |
|---|---|
| Water for injection | qs |
| pH adjusted to 5.0 | |

Example G:

| Terlipressin acetate | 100 | μg/ml |
|---|---|---|
| Sodium benzoate | 4.5 | mM |
| Sodium chloride | 150 | mM |
| Water for injection | qs | |
| pH adjusted to 5.0 | | |

Example H:

| Terlipressin acetate | 100 | μg/ml |
|---|---|---|
| Sodium benzoate | 3 | mM |
| Sodium chloride | 140 | mM |
| Glycine | 10 | mM |
| Water for injection | qs | |
| pH adjusted to 5.0 | | |

Example I:

| Terlipressin acetate | 100 | μg/ml |
|---|---|---|
| Sodium acetate | 1 | mM |
| Sodium chloride | 150 | mM |
| Water | qs | |
| pH adjusted to 4.5 | | |

Example J:

| Terlipressin acetate | 100 | μg/ml |
|---|---|---|
| Sodium acetate | 3 | mM |
| Sodium chloride | 150 | mM |
| Water for injection | qs | |
| pH adjusted to 4.5 | | |

Example K:

| Terlipressin acetate | 100 | μg/ml |
|---|---|---|
| Sodium acetate | 4.5 | mM |
| Sodium chloride | 150 | mM |
| Water for injection | qs | |
| pH adjusted to 4.5 | | |

Example L:

| Terlipressin acetate | 100 | μg/ml |
|---|---|---|
| Sodium acetate | 3 | mM |
| Sodium chloride | 140 | mM |
| Glycine | 10 | mM |
| Water for injection | qs | |
| pH adjusted to 4.5 | | |

The stability of the formulations is determined using the visual assessment and RP-HPLC methods (see General Methods) following incubation at 40° C. for 2, 4 and 8 weeks.

The stability of the formulations is determined using a visual assessment and RP-HPLC methods (see General Methods) following incubation at 25° C. for 4 8 and 12 weeks.

The stability of the formulations is determined using a visual assessment and RP-HPLC methods (see General Methods) following incubation at 2-8° C. for 8 and 12 weeks.

### Example 2: Effect of Buffer Concentration on Stability of Terlipressin

The effect of buffer concentration on the rate of impurity formation in compositions of terlipressin (as acetate) was investigated using the RP-HPLC method described in General Methods, following storage at 40° C. and 50° C. The effect was investigated in the presence of mannitol (300 mM) and in the presence of sodium chloride (150 mM) as tonicity modifiers. Three buffers were tested: acetate, citrate and lactate. The pH of all compositions was 4.5. The results are shown in Table 1.

TABLE 1
Increase in impurity level in compositions of terlipressin following storage at
40° C. and 50° C. All compositions were adjusted to pH 4.5.

| | | | | | | Increase in % impurity | | | |
|---|---|---|---|---|---|---|---|---|---|
| Example. | Acetate | citrate | lactate | Mannitol | NaCl | 40° C. | 40° C. | 50° C. | 50° C. |
| No. | (mM)* | (mM) | (mM) | (mM) | (mM) | (2 weeks) | (4 weeks) | (2 weeks) | (4 weeks) |
| 2-1 | 0.2 | | | 300 | | 0.07 | 0.26 | 0.43 | 1.20 |
| 2-2 | 1 | | | 300 | | 0.10 | 0.32 | 0.53 | 1.33 |
| 2-3 | 3 | | | 300 | | 0.17 | 0.36 | 0.68 | 1.59 |
| 2-4 | 4.5 | | | 300 | | 0.18 | 0.37 | 0.72 | 1.59 |
| 2-5 | 5 | | | 300 | | 0.21 | 0.50 | 0.77 | 1.83 |
| 2-6 | 10 | | | 300 | | 0.26 | 0.71 | 0.94 | 2.14 |
| 2-7 | 20 | | | 300 | | 0.38 | 0.90 | 1.27 | 3.00 |
| 2-8 | 0.2 | 0.8 | | 300 | | 0.32 | 0.86 | 0.94 | 2.05 |
| 2-9 | 0.2 | 2.8 | | 300 | | 0.96 | 2.14 | 1.45 | 3.39 |
| 2-10 | 0.2 | 4.3 | | 300 | | 1.22 | 3.08 | 1.69 | 4.00 |
| 2-11 | 0.2 | 4.8 | | 300 | | 1.51 | 3.33 | 1.90 | 4.44 |
| 2-12 | 0.2 | 9.8 | | 300 | | 2.16 | 4.87 | 3.55 | 9.12 |
| 2-13 | 0.2 | 19.8 | | 300 | | 3.55 | 7.92 | 7.30 | 19.54 |
| 2-14 | 0.2 | | 0.8 | 300 | | 0.12 | 0.29 | 0.55 | 1.34 |
| 2-15 | 0.2 | | 2.8 | 300 | | 0.19 | 0.46 | 0.74 | 1.60 |
| 2-16 | 0.2 | | 4.3 | 300 | | 0.24 | 0.51 | 0.75 | 1.77 |
| 2-17 | 0.2 | | 4.8 | 300 | | 0.29 | 0.68 | 0.86 | 1.98 |
| 2-18 | 0.2 | | 9.8 | 300 | | 0.44 | 0.92 | 1.12 | 2.43 |
| 2-19 | 0.2 | | 19.8 | 300 | | 0.56 | 1.19 | 1.33 | 2.90 |
| 2-20 | 1 | | | | 150 | 0.15 | 0.40 | 0.59 | 1.38 |
| 2-21 | 3 | | | | 150 | 0.18 | 0.41 | 0.78 | 1.69 |
| 2-22 | 4.5 | | | | 150 | 0.20 | 0.56 | 0.79 | 1.83 |
| 2-23 | 5 | | | | 150 | 0.25 | 0.67 | 0.87 | 2.01 |
| 2-24 | 10 | | | | 150 | 0.32 | 0.89 | 1.00 | 2.47 |
| 2-25 | 20 | | | | 150 | 0.50 | 1.11 | 1.49 | 3.50 |
| 2-26 | 0.2 | 0.8 | | | 150 | 0.26 | 0.69 | 0.83 | 1.80 |
| 2-27 | 0.2 | 2.8 | | | 150 | 0.87 | 1.94 | 1.19 | 2.41 |
| 2-28 | 0.2 | 4.3 | | | 150 | 1.12 | 2.37 | 1.44 | 3.12 |
| 2-29 | 0.2 | 4.8 | | | 150 | 1.37 | 2.98 | 1.71 | 3.97 |
| 2-30 | 0.2 | 9.8 | | | 150 | 1.68 | 4.12 | 3.01 | 8.86 |
| 2-31 | 0.2 | 19.8 | | | 150 | 2.99 | 6.88 | 6.18 | 15.94 |
| 2-32 | 0.2 | | 0.8 | | 150 | 0.13 | 0.37 | 0.56 | 1.34 |
| 2-33 | 0.2 | | 2.8 | | 150 | 0.18 | 0.40 | 0.78 | 1.66 |
| 2-34 | 0.2 | | 4.3 | | 150 | 0.23 | 0.56 | 0.80 | 1.99 |
| 2-35 | 0.2 | | 4.8 | | 150 | 0.30 | 0.71 | 0.85 | 2.06 |
| 2-36 | 0.2 | | 9.8 | | 150 | 0.41 | 0.89 | 1.18 | 2.45 |
| 2-37 | 0.2 | | 19.8 | | 150 | 0.52 | 1.10 | 1.45 | 3.19 |

*includes 0.2 mM contribution of acetate counterion of terlipressin

Formulations containing a low buffer concentration in the presence of mannitol or sodium chloride as tonicity modifier had good stability. It was shown that increasing the buffer concentration resulted in stability impairment. The effect was demonstrated with all three buffers tested and was particularly strong for citrate. The nature of the tonicity modifier had only a relatively small effect on the stability of terlipressin. Formulations containing a low concentration of acetate or lactate as buffer appeared to have particularly good stability, particularly acetate as buffer.

### Example 3: Effect of Amino Acids on the Stability of Terlipressin in Compositions Comprising 3 mM Acetate Buffer

The effect of amino buffer concentration on the rate of impurity formation in compositions of terlipressin (as acetate) was investigated using the RP-HPLC method described in General Methods, following storage at 40° C. and 50° C. The effect was investigated in the presence of mannitol (300 mM) and in the presence of sodium chloride (150 mM) as tonicity modifiers. All compositions comprised 3 mM acetate and were adjusted to pH 4.5. The effect of proline, glycine and methionine was tested at 5 mM and 30 mM concentrations. The results are shown in Table 2.

TABLE 2
Increase in impurity level in compositions of terlipressin following storage at
40° C. and 50° C. All compositions were adjusted to pH 4.5.

| | | | | | | | Increase in % impurity | | | |
|---|---|---|---|---|---|---|---|---|---|---|
| Comp. | Acetate | Mannitol | NaCl | Proline | Glycine | Methionine | 40° C. | 40° C. | 50° C. | 50° C. |
| No. | (mM)* | (mM) | (mM) | (mM) | (mM) | (mM) | (2 weeks) | (4 weeks) | (2 weeks) | (4 weeks) |

| | | | | | | | | | |
|---|---|---|---|---|---|---|---|---|---|
| 38 | 3 | 300 | | | | 0.19 | 0.46 | 0.74 | 1.60 |
| 39 | 3 | 300 | 5 | | | 0.10 | 0.29 | 0.59 | 1.47 |
| 40 | 3 | 300 | 30 | | | 0.21 | 0.49 | 0.69 | 1.82 |
| 41 | 3 | 300 | | 5 | | 0.11 | 0.31 | 0.59 | 1.50 |
| 42 | 3 | 300 | | 30 | | 0.17 | 0.55 | 0.64 | 1.96 |
| 43 | 3 | 300 | | | 5 | 0.11 | 0.27 | 0.55 | 1.42 |
| 44 | 3 | 300 | | | 30 | 0.10 | 0.47 | 0.56 | 1.63 |
| 45 | 3 | 150 | | | | 0.18 | 0.41 | 0.78 | 1.69 |
| 46 | 3 | 150 | 5 | | | 0.13 | 0.37 | 0.62 | 1.63 |
| 47 | 3 | 150 | 30 | | | 0.21 | 0.44 | 0.79 | 1.79 |
| 48 | 3 | 150 | | 5 | | 0.12 | 0.35 | 0.65 | 1.62 |
| 49 | 3 | 150 | | 30 | | 0.14 | 0.49 | 0.68 | 1.83 |
| 50 | 3 | 150 | | | 5 | 0.12 | 0.33 | 0.63 | 1.59 |
| 51 | 3 | 150 | | | 30 | 0.13 | 0.49 | 0.63 | 1.70 |

*includes 0.2 mM contribution of acetate counterion of terlipressin

It was shown that the addition of 5 mM amino acid resulted in improvement stability of terlipressin, both using mannitol and using sodium chloride as a tonicity modifier. Increasing the amino acid concentration to 30 mM did not provide an additional stabilisation effect. In fact, in most cases, the use of 30 mM amino acid led to a slight stability impairment compared with using 0 mM or 5 mM amino acid.

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

All patents, patent applications and references mentioned throughout the specification of the present invention are herein incorporated in their entirety by reference.

The invention embraces all combinations of preferred and more preferred groups and suitable and more suitable groups and embodiments of groups recited above.

1. An aqueous solution composition having a pH in the range 4.0-6.0 comprising
terlipressin or a salt thereof and
optionally one or more buffers having at least one ionisable group with a pKₐ in the range 3.0 to 7.0 and which pKₐ is within 1 pH unit of the pH of the composition
tonicity
and wherein the one or more buffers are present in the composition at a total concentration of 0-5 mM.

2. The aqueous solution composition according to claim 1, wherein the concentration of terlipressin or salt thereof is 0.001-50 mg/ml, such as 0.01-10 mg/ml, 0.01-5 mg/ml, 0.01-3 mg/ml, 0.01-2 mg/ml, 0.01-1 mg/ml or 0.1-1 mg/ml.

3. The aqueous solution composition according to claim 1, which comprises one or more buffers and wherein the total concentration of the one or more buffers in the composition is 0.1-5 mM, such as 0.1-4 mM, 0.1-3 mM, 0.1-2 mM, 0.1-1 mM, 0.1-0.5 mM, 0.1-0.4 mM, 0.1-0.3 mM or 0.1-0.2 mM; and/or wherein the total concentration of the one or more buffers in the composition is ≤5 mM, such as ≤4 mM, ≤3 mM, ≤2 mM, ≤1 mM, ≤0.5 mM, ≤0.4 mM, ≤0.3 mM, ≤0.2 mM or ≤0.1 mM.

4. (canceled)

5. The aqueous solution composition according to claim 1 which is substantially free of a buffer.

6. The aqueous solution composition according to claim 1, which comprises one or more buffers selected from histidine, maleate, glyoxylate, aspartame, glucuronate, aspartate, glutamate, tartrate, gluconate, lactate, glycolic acid, adenine, succinate, ascorbate, benzoate, phenylacetate, gallate, cytosine, p-aminobenzoic acid, sorbate, acetate, propionate, alginate, urate, 2-(N-morpholino)ethanesulphonic acid, bicarbonate, bis(2-hydroxyethyl) iminotris(hydroxymethyl)methane, N-(2-acetamido)-2-iminodiacetic acid, 2-[(2-amino-2-oxoethyl)amino]ethanesulphonic acid, piperazine and N,N′-bis(2-ethanesulphonic acid) and salts thereof, and combinations thereof.

7. (canceled)

8. The aqueous solution composition according to claim 1, which further comprises an amino acid.

9. The aqueous solution composition according to claim 8, wherein the amino acid is selected from glycine, proline, methionine, arginine, lysine, aspartic acid, glutamic acid and histidine, especially wherein the amino acid is selected from glycine, proline, methionine, arginine and lysine, and in particular wherein the amino acid is glycine, proline or methionine e.g. methionine.

10. (canceled)

11. The aqueous solution composition according to claim 8, wherein the amino acid is present at a concentration of 1-200 mM, such as 1-100 mM, 1-50 mM, 1-20 mM, 1-10 mM, 1-5 mM, 1-4 mM, 1-3 mM or 1-2 mM.

12. An aqueous solution composition having a pH in the range 4.0-6.0 and comprising:
terlipressin or a salt thereof;
one or more buffers having at least one ionisable group with a pKₐ in the range 3.0 to 7.0 and which pKₐ is within 1 pH unit of the pH of the composition; and
an amino acid which does not comprise an ionisable groups with a pKₐ within 1 pH unit of the pH of the composition; and
wherein the one or more buffers are present in the composition at a total concentration of 0.5 to 5 mM e.g. 0.5 to 3 mM.

13. The aqueous solution composition according to claim 12,
wherein the amino acid is selected from methionine, glycine and proline; and/or
wherein the amino acid is present at a concentration of 2-20 mM.

14. (canceled)

15. The aqueous solution composition according to claim 12, wherein one or more buffers is selected from histidine, maleate, tartrate, lactate, benzoate, acetate and bicarbonate, particularly lactate and acetate especially acetate.

16. (canceled)

17. The aqueous solution composition according to claim 1, which further comprises an uncharged tonicity modifier selected from glycerol, 1,2-propanediol, mannitol, sorbitol, sucrose, trehalose, lactose, PEG300 and PEG400, and in particular is selected from mannitol and sorbitol.

18. The aqueous solution composition according to claim 17, wherein the uncharged tonicity modifier is present at a concentration of 50-1000 mM, such as 200-500 mM, or about 300 mM.

19. The aqueous solution composition according to claim 1, which further comprises a charged tonicity modifier selected from sodium chloride and sodium sulphate.

20. (canceled)

21. The aqueous solution composition according to claim 19, wherein the charged tonicity modifier is present at a concentration of 25-500 mM, such as 50-250 mM, or about 150 mM.

22. The aqueous solution composition according to claim 1, which further comprises a non-ionic surfactant selected from an alkyl glycoside, a polysorbate, an alkyl ether of polyethylene glycol, a block copolymer of polyethylene glycol and polypropylene glycol, and an alkylphenyl ether of polyethylene glycol, and in particular a polysorbate such as polysorbate 20 or polysorbate 80.

23. (canceled)

24. (canceled)

25. The aqueous solution composition according claim 22, wherein the non-ionic surfactant is present at a concentration of 10-2000 μg/ml, such as 50-1000 μg/ml, 100-500 μg/ml or about 200 μg/ml.

26. The aqueous solution composition according to claim 1, which additionally comprises a preservative, which is a phenolic or benzylic preservative selected from phenol, m-cresol, chlorocresol, benzyl alcohol, propyl paraben and methyl paraben.

27. (canceled)

28. The aqueous solution composition according to claim 1, wherein terlipressin is employed as terlipressin acetate.

29. (canceled)

30. An aqueous solution composition according to claim 1, which is a pharmaceutical composition.

31. (canceled)

32. A method of treatment of hypotension, septic shock, esophageal varices or hepatorenal syndrome which comprises administering to a patient in need thereof a therapeutically effective amount of an aqueous solution composition according to claim 1.
